(19) United States
(12) Patent Application Publication
(10) Pub. No.: US 2025/0082192 A1
(43) Pub. Date: Mar. 13, 2025

(54) ENDOTRACHEAL DEVICE

(71) Applicant: Robert Salasidis, Saint-Laurent (CA)

(72) Inventor: Robert Salasidis, Saint-Laurent (CA)

(21) Appl. No.: 18/243,952

(22) Filed: Sep. 8, 2023

(51) Int. Cl. A61B 1/267; A61B 1/00; A61M 16/04

(52) U.S. Cl. CPC A61B 1/2676; A61B 1/00082; A61B 1/00094; A61B 1/00137; A61B 1/00154; A61M 16/0434; A61M 16/0463; A61B 2017/22079; A61M 2039/1077

(57) ABSTRACT

An example endotracheal device includes: a tube having an end surface at a first end of the tube, the end surface having a rounded edge and configured to be aligned with a target obstruction; an adaptor disposed at a second end of the tube, the adaptor having: a bronchoscope connector comprising an aperture configured to admit a bronchoscope through the second end along a length of the tube to the first end of the tube to guide the tube to the target obstruction; and a suction port configured to connect to a suction device to apply suction to the tube to engage the target obstruction at the end surface.

### FIELD

The specification relates generally to endotracheal devices, and more particularly to an endotracheal device to remove a target obstruction.

### BACKGROUND

Clots or other obstructions can occur in the trachea of a subject and can be very dangerous due to the obstruction of the airway. Accordingly, such obstructions should be removed in a timely fashion.

### SUMMARY

According to an aspect of the present specification an example endotracheal device includes: endotracheal device comprising: a tube having an end surface at a first end of the tube, the end surface having a rounded edge and configured to be aligned with a target obstruction; an adaptor disposed at a second end of the tube, the adaptor having: a bronchoscope connector comprising an aperture configured to admit a bronchoscope through the second end along a length of the tube to the first end of the tube to guide the tube to the target obstruction; and a suction port configured to connect to a suction device to apply suction to the tube to engage the target obstruction at the end surface.

According to another aspect of the present specification, an example method includes: administering an endotracheal device having an end surface with a rounded edge at a first end into an endotracheal system of a subject; advancing a bronchoscope through the endotracheal device to a target obstruction in the endotracheal system of the subject; advancing the endotracheal device along the bronchoscope to align the end surface against the target obstruction; retracting the bronchoscope; applying suction to the endotracheal device to engage the target obstruction; and retracting the endotracheal device to remove the target obstruction from the endotracheal system of the subject.

### BRIEF DESCRIPTION OF DRAWINGS

Implementations are described with reference to the following figures, in which:

FIG. 1 depicts a schematic diagram of an example endotracheal device, in accordance with the present disclosure.

FIG. 2 depicts a schematic diagram of another example endotracheal device, in accordance with the present disclosure.

FIG. 3 depicts a schematic diagram of an example adaptor for use with the endotracheal device of FIG. 1 or FIG. 2.

FIGS. 4A and 4B depict schematic diagrams of another example adaptor for use with the endotracheal device of FIG. 1. or FIG. 2.

FIG. 5 depicts a flowchart of an example method of using an endotracheal device to remove a target obstruction from the endotracheal system of a subject, in accordance with the present disclosure.

FIGS. 6A-D depict schematic diagrams of exemplary performances of blocks 515 to 535 of the method of FIG. 5.

### DETAILED DESCRIPTION

Obstructions in the trachea or endotracheal system of a subject may be sufficiently acute as to require immediate removal. The obstruction, depending on the cause, may, in some cases, be removed with nasotracheal suction, or suction with standard catheters via an endotracheal tube. A bronchoscope may also be used for targeted suctioning of a mucous plug or possibly other types of obstruction. A problem arises when the secretion, blood or other type of obstruction is of sufficient size, that the small diameter suction port on the bronchoscope, or a nasotracheal catheter is not able to remove it. Transporting the subject to an operating room for rigid bronchoscopy may be too time-intensive and may negatively affect the subject's outcome. Accordingly, in acute situations, a standard endotracheal tube may be administered into the subject's endotracheal system to the obstruction, and then suction applied to engage the obstruction for its removal. However, a standard endotracheal tube includes a Murphy's eye (i.e., a hole near the end to allow additional air ventilation) which reduces the effectiveness of the suction. Trimming the standard endotracheal tube to eliminate the Murphy's eye reduces the length of the tube and also creates sharp or jagged edges and/or protrusions which may irritate or damage the endotracheal system of the subject as the standard endotracheal tube is advanced and retracted to retrieve the obstruction. Additionally, it may be difficult to locate the obstruction by simple intubation, particularly when the obstruction occurs in an opposing direction to the natural curvature of the endotracheal tube (such as left sided obstructions).

According to the present disclosure, an example endotracheal device may facilitate removal of a target obstruction in the endotracheal system of a subject. In particular, a tube of the endotracheal device is administered to the endotracheal system. An adaptor of the tube includes a bronchoscope connector, to allow a bronchoscope to be inserted into the endotracheal device. The bronchoscope may be advanced using the bronchoscope camera to locate the target obstruction in the endotracheal system. The endotracheal device may then be advanced along the bronchoscope, such that the bronchoscope acts as a guide to align the tube with or against the target obstruction. The adaptor of the tube, and in particular, the bronchoscope connector, therefore allows the bronchoscope to be advanced through and retracted from the tube.

Advantageously, the tube has an end surface configured to be aligned against the target obstruction. Further, the end surface has a rounded edge, for example extending between the end surface and the exterior surface of the tube, to facilitate movement of the tube within the endotracheal system of the subject and to reduce sharp corners or other protrusions which may injure the trachea as the tube is advanced and retracted.

Once the end surface has been aligned against the target obstruction, the bronchoscope is at least partially retracted through the tube, and suction is applied at a suction port to the tube to engage the target obstruction. Suction is then maintained as the endotracheal device is retracted from the endotracheal system to remove the target obstruction. The diameter of the tube, being larger than a standard bronchoscope, results in a correspondingly larger suction force. This results in a higher success rates of removal as well as quicker removal of the obstruction.

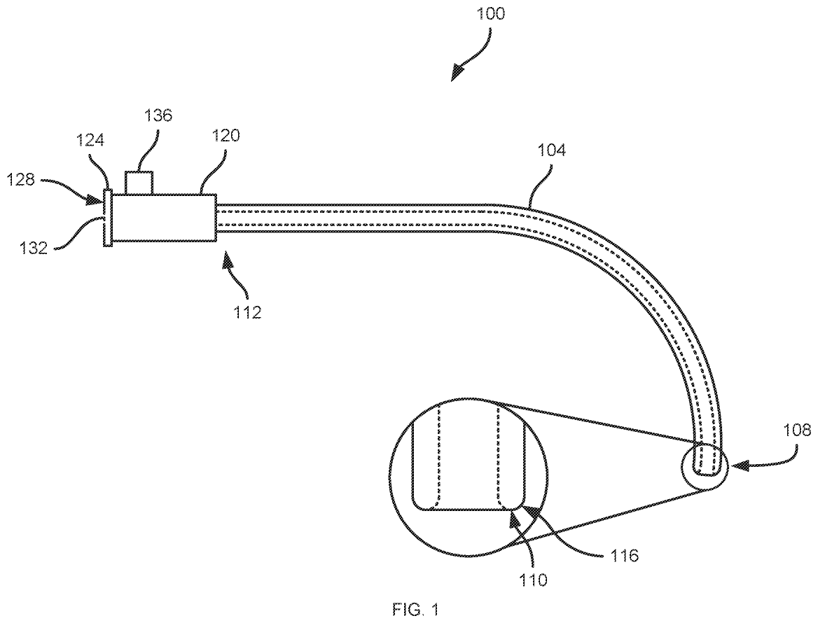

FIG. 1 depicts an endotracheal device 100 in accordance with the present disclosure. The endotracheal device 100 is generally configured to be used in the endotracheal system of a subject to retrieve and remove a target obstruction (e.g., a mass, a clot, or the like) in the subject's endotracheal system.

The endotracheal device 100 includes a tube 104 having a length L extending between a first end 108 and a second end 112. In particular, both the first end 108 and the second end 112 are open and the tube 104 defines a hollow interior passageway along the length L between the open first end 108 and the open second end 112.

The tube 104 is generally configured to be inserted into the subject's endotracheal system (i.e., via the nose or mouth of the subject) and may be similar to a standard endotracheal tube. In particular, the tube 104 may be flexible to allow maneuvering through the subject's endotracheal system and formed of a suitable material for the health and safety of the subject. The tube 104 may have a suitable length L to extend the first end 108 into a subject's endotracheal system, while still having the second end 112 be accessible. For example, the tube 104 may be about 25 cm to about 30 cm. In other examples, other suitable lengths are also contemplated. For example, the tube 104 may be shorter if the subject is a child.

The tube 104 may further be configured to receive a bronchoscope along the length L to allow the bronchoscope to guide the tube 104 to the target obstruction, as will be described further herein. Accordingly, the tube 104 may have a suitable diameter to accommodate a standard bronchoscope. For example, the tube 104 may have a diameter of about 6.5 mm to about 8 mm. In other examples, the tube 104 may have a wider or a narrower diameter.

In contrast to a standard endotracheal tube, the tube 104 also has a rounded edge 116 at the first end 108. That is, the first end 108 includes an end surface 110. The end wall 110 may preferably be substantially perpendicular to the length L of the tube 104 but may also be at any suitable angle. The first end 108 may further include an inner edge from an interior surface of the tube 104 to the end surface 110 of the tube 104, and an outer edge from the end surface 110 of the tube 104 to an exterior surface of the tube 104. At least one of the inner edge and the outer edge, and preferably both, is rounded to form a smooth, curved surface from the end surface 110 to either the interior or exterior surface of the tube 104. That is, at least one boundary of the end surface 110 forms the rounded edge 116.

More particularly, the rounded edge 116 may be defined by any curved, continuous transitional surface from the end surface 110 to the interior or exterior surface of the tube 104. The rounded edge 116 may, for example, follow the curve of a circle or an ellipse having a non-zero diameter, and preferably, a diameter of about the width of the wall of the tube 104 (i.e., a diameter of about a closest distance between the interior surface and the exterior surface of the tube 104). In other examples, the rounded edge 116 may follow any other suitable curve.

In particular, the rounded edge 116 is configured to reduce any rough portions, protrusions, irregularities, or the like at the first end 108 of the tube 104, so as to allow the endotracheal device 100 to be inserted into, advanced in and retracted from the subject's endotracheal system with reduced risk of damage by the first end 108.

The first end 108, and more particularly the end surface 110 is configured to be aligned with or positioned against the target obstruction to engage the target obstruction, as will further be described herein.

The rounded edge 116 may preferably be integral with the tube 104; that is, the rounded edge 116 may be formed by smoothing the material of the tube 104 to remove rough or jagged edges. In other examples, a separate endpiece may be applied at the first end 108 of the tube 104 to form the end surface 110 and the rounded edge 116. For example, the endpiece may be formed of a different material, including an elastic or deformable material to allow the end surface 110 to substantially conform to the target obstruction. Further, a transition region between the endpiece and the tube 104 may similarly be adapted to have a smooth, continuous surface and/or transition.

The endotracheal device 100 further includes an adaptor 120 disposed at the second end 112 of the tube 104. The adaptor 120 is generally configured to serve as an intermediate between other devices and the tube 104 and hence may be configured to maintain fluid communication between the tube 104 and the components of the adaptor 120 for connection to other devices.

In particular, the adaptor 120 includes a bronchoscope connector 124 configured to admit a bronchoscope into the length L of the tube 104. In particular, the bronchoscope connector 124 may include a membrane 128 having an aperture 132. Thus, the bronchoscope may be admitted through the bronchoscope connector 124 via the aperture 132. The membrane 128 may be flexible to allow the bronchoscope to be advanced and retracted through the aperture 132 while maintaining a substantial seal around the bronchoscope. That is, the bronchoscope connector 124 may further be configured to allow the bronchoscope to be advanced within the endotracheal system of the subject to the target obstruction by admitting the bronchoscope along the length L of the tube 104 and out the first end 108. Upon locating the target obstruction with the bronchoscope, the bronchoscope connector 124 is further configured to allow the endotracheal device 100 to be advanced along the bronchoscope, to allow the bronchoscope to guide the tube 104, and more particularly, the end surface 110, to be positioned against the target obstruction. The bronchoscope connector 124 is further configured to allow the bronchoscope to be retracted through the tube 104 towards the second end 112.

The adaptor 120 further includes a suction port 136 configured to connect the tube 104 to a suction device (e.g., a suction machine such as aspirator or similar) configured to apply suction to the tube 104. In particular, when the end surface 110 of the endotracheal device 100 is positioned against the target obstruction, and the bronchoscope has been retracted through the tube 104, the suction device may be configured to apply suction to the tube 104 to engage the target obstruction at the end surface 110. That is, the suction applied to the tube via the suction port 136 is configured to draw the target obstruction to the end surface 110 and to maintain the target obstruction at the rounded edge 116. The endotracheal device 100 may then be removed from the subject's endotracheal system while maintaining suction at the suction port 136 to engage the target obstruction for removal of the target obstruction with the endotracheal device 100.

Accordingly, in contrast with a standard endotracheal tube, the tube 104 lacks a Murphy's eye, or an additional air vent hole, near the first end 108 of the tube 104 as such a ventilation hole would reduce the effectiveness of the suction to engage and maintain the target obstruction for removal of the target obstruction.

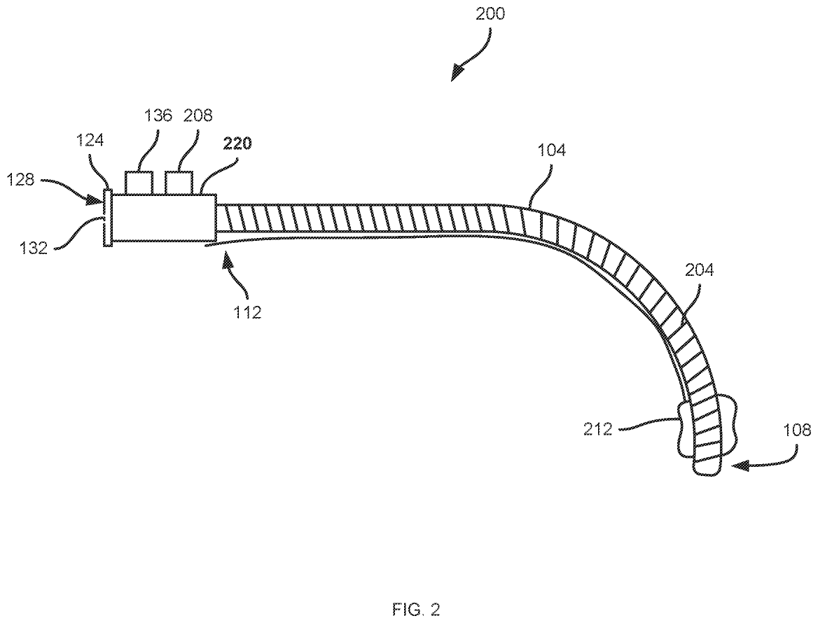

In other examples, other features and components may be applied to the endotracheal device in accordance with the present disclosure. For example, FIG. 2 depicts another example endotracheal device 200. The endotracheal device 200 is similar to the endotracheal device 100 and is generally configured to be used in the endotracheal system of a subject to retrieve and remove a target obstruction in the subject's endotracheal system, and hence like numbers are used to depict like elements.

In particular, the endotracheal device 200 further includes armoring 204 on the tube 104. For example, the armoring 204 may be formed in a spiral or other suitable geometry or configuration along the length L of the tube 104. The armoring 204 is configured to reinforce the structural integrity of the tube 104 so as to reduce the likelihood of kinks or sharp bends in the tube 104 which may obstruct the passageway of the tube 104, while still allowing flexibility and maneuverability of the tube 104 in the endotracheal system of the subject. That is, the armoring 204 may maintain the passageway open through the length L of the tube 104. The armoring 204 may be made integrally with the tube 104 or may be a separate component applied to the tube 104.

The endotracheal device 200 further includes an adaptor 220 including the bronchoscope connector 124, the suction port 136, and a ventilation port 208. The ventilation port 208 is configured to connect the tube 104 to a ventilator, for example to ventilate the subject through the endotracheal device 200. To further facilitate the ventilation, the endotracheal device 200 may further include an endotracheal cuff 212 configured to be inflated to seal the airway in which the endotracheal device 200 is inserted to maintain the position of the endotracheal device 200 within the endotracheal system of the subject. Accordingly, when the endotracheal cuff 212 is inflated the subject is ventilated via the ventilation port 208. This allows more time to position the tube over the obstruction, while allowing periodic ventilation to occur between tube manipulations.

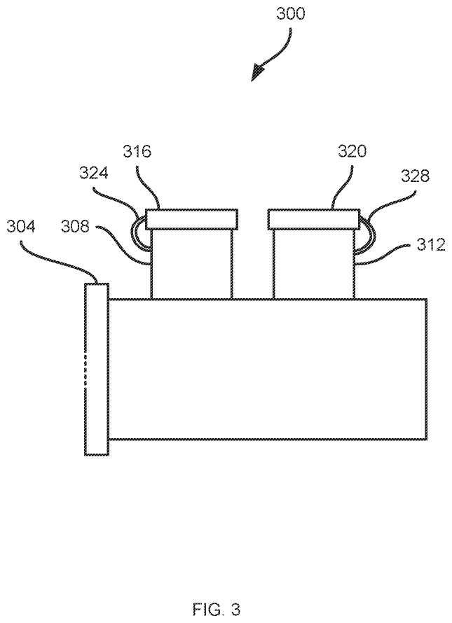

FIG. 3 depicts an example adaptor 300 which may be used, for example, in place of the adaptor 120 or the adaptor 220. The adaptor 300 includes a bronchoscope connector 304, a suction port 308, and a ventilation port 312. The adaptor 300 further includes a suction port cap 316 and a ventilation port cap 320.

The suction port cap 316 is configured to selectively cover the suction port 308, for example when the suction port 308 is not in use. The suction port cap 316 may be configured to be secured to the suction port 308 via any suitable means, such as via a screw-on mechanism (i.e., via mating threads), an interference fit, a snap fit, or other suitable mechanisms. Further, the suction port cap 316 is removable from the suction port 308 to allow a suction device to be connected to the suction port 308. In some examples, the suction port cap 316 may include a lead 324 or other suitable connector to tether the suction port cap 316 to the adaptor 300 when the suction port cap 316 is removed from the suction port 308.

The ventilation port cap 320 is configured to selectively cover the ventilation port 312, for example when the ventilation port 312 is not in use. The ventilation port cap 320 may be configured to be secured to the ventilation port 312 via any suitable means, such as via a screw-on mechanism (i.e., via mating threads), an interference fit, a snap fit, or other suitable mechanisms. Further, the ventilation port cap 320 is removable from the ventilation port 312 to allow a ventilator to be connected to the ventilation port 312. In some examples, the ventilation port cap 320 may include a lead 328 or other suitable connector to tether the ventilation port cap 320 to the adaptor 300 when the ventilation port cap 320 is removed from the ventilation port 312.

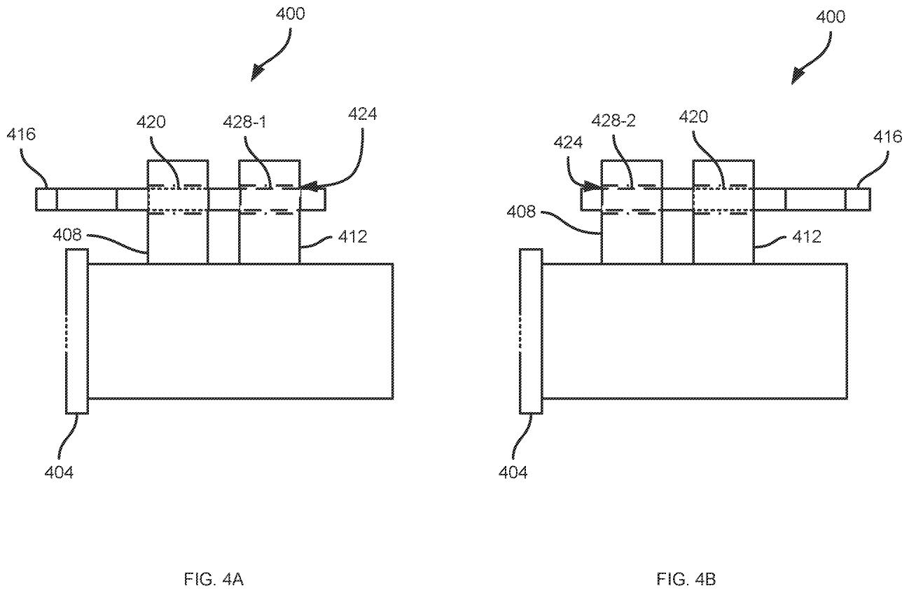

FIG. 4A depicts another example adaptor 400 which may be used, for example, in place of the adaptor 120, or the adaptor 220. The adaptor 400 similarly includes a bronchoscope connector 404, a suction port 408, and a ventilation port 412. The adaptor 400 further includes a port selector 416.

The port selector 416 is configured to selectively cover the suction port 408 or the ventilation port 412 and allow access to the other port. Thus, the port selector 416 may be a moveable piece configured to move with respect to the suction port 408 and the ventilation port 412 to selectively cover one of the ports 408 and 412. Preferably, the port selector 416 mutually exclusively select either the suction port 408 or the ventilation port 412, such that at most one of the suction port 408 or the ventilation port 412 accessible at a given time.

For example, as illustrated in FIG. 4A, the port selector 416 includes an opening 420. The port selector 416 is configured to slide in a slot 424 to align the opening 420 with either the suction port 408 or the ventilation port 412. In particular, the port selector 416 may slide between a first position, in which the opening 420 is aligned with the suction port 408, as illustrated in FIG. 4A, and a second position, in which the opening 420 is aligned with the ventilation port, as illustrated in FIG. 4B. When the opening 420 is aligned with the suction port 408, a first blocking portion 428-1 of the port selector 416 blocks the ventilation port 412. Similarly, when the opening 420 is aligned with the ventilation port 412, a second blocking portion 428-2 of the port selector 416 blocks the suction port 408.

In some examples, the port selector 416 and the slot 424 may include position selecting mechanisms, such as corresponding protrusions and notches, or the like, which align the opening 420 with either the suction port 408 or the ventilation port 412. In some examples, the port selector 416 may be biased (e.g., via a spring or the like) to be aligned with a default port of the suction port 408 and the ventilation port 412. Further, the port selector 416 may protrude from the slot 424 and may include a tab, a grip, or the like, to facilitate movement between the first position and the second position by a user.

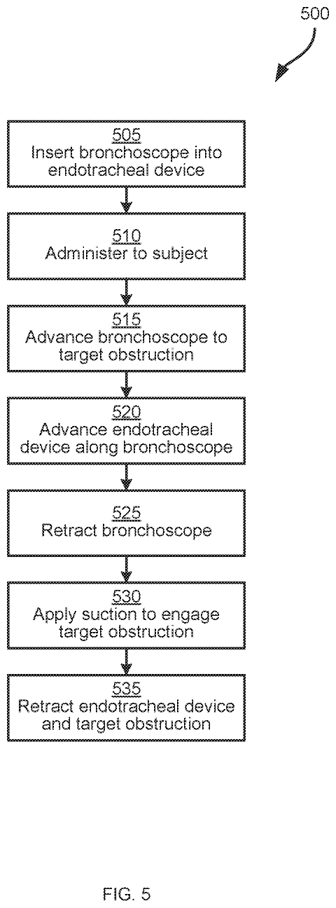

Turning now to FIG. 5, an example method 500 of removing a target obstruction from the endotracheal system of a subject is depicted. The method 500 may be performed, for example, using the endotracheal device 100 or 200, and/or with the adaptors 300 or 400. In other examples, the method 500 may be performed using other suitable devices or systems.

At block 505, an endotracheal device, such as the endotracheal device 100 or 200, may receive a bronchoscope therein. That is, a user may insert a bronchoscope into the endotracheal device via a bronchoscope connector, and more particularly, through an aperture at a second end along the length L of a tube of the endotracheal device. In particular, a camera end of the bronchoscope is inserted into the endotracheal device towards a first end of the tube.

At block 510, the endotracheal device with the bronchoscope contained therein is administered into the endotracheal system of the subject. For example, the endotracheal device may be inserted through the nose or the mouth of the subject. More particularly, the first end of the tube, together with the camera end of the bronchoscope, is inserted into the endotracheal system of the subject. In other examples, blocks 505 and blocks 510 may be performed in reverse order. That is, the endotracheal device may be administered into the endotracheal system of the subject, and subsequently, the bronchoscope may be inserted into the endotracheal device in the endotracheal system of the subject.

At block 515, the bronchoscope is advanced within the endotracheal system of the subject to the target obstruction. In particular, the user may use a viewing end of the bronchoscope to observe the camera view and maneuver the bronchoscope to the target obstruction. Thus, the bronchoscope may be extended to and through the first end of the tube until the target obstruction is located.

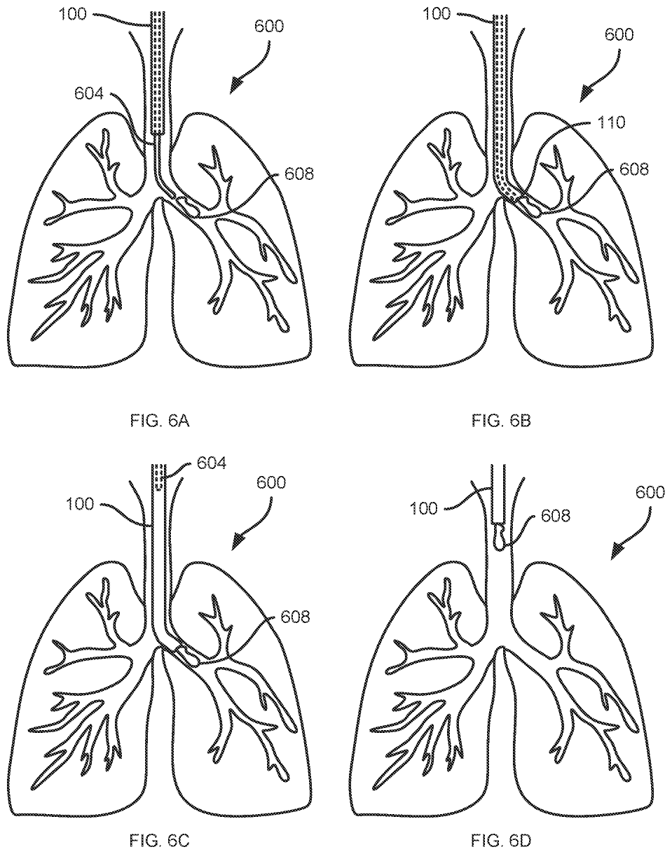

For example, referring to FIG. 6A, an example schematic diagram of the use of the endotracheal device 100 in an endotracheal system 600 of a subject at block 515 of the method 500 is depicted. In particular, the endotracheal device 100 is inserted at least partially into the endotracheal system 600 of the subject with a bronchoscope 604. Upon advancing the tube 104 to a fork or junction within the endotracheal system 600, the bronchoscope 604 may be advanced past the first end 108 and maneuvered to locate a target obstruction 608. For example, a camera of the bronchoscope 604 may be used to locate and advance the bronchoscope 604 to the target obstruction 608.

Returning to FIG. 5, at block 520, the endotracheal device is advanced along the bronchoscope to the target obstruction. That is, the bronchoscope acts as a guide for the endotracheal device, and more particularly, the tube, to reach the target obstruction. Using the bronchoscope as a guide may facilitate maneuvering of the tube, in particular when the target obstruction is located in a direction or along a path opposite a natural curvature of the tube. Further, using the bronchoscope as a guide may improve the accuracy and speed of aligning the tube, and more particularly, an end surface of the tube, with the target obstruction.

For example, referring to FIG. 6B, an example schematic diagram of the use of the endotracheal device 100 in the endotracheal system 600 at block 520 of the method 500 is depicted. In particular, after the bronchoscope 604 is advanced to the target obstruction 608, the tube 104 is advanced along the bronchoscope 604 until the end surface 110 abuts or is positioned against the target obstruction 608. That is, the bronchoscope 604 guides the tube 104 to align the end surface 110 with the target obstruction 608.

Further, returning to FIG. 5, in some examples, during the method 500, for example, prior to advancing the end surface to align with the target obstruction, the subject may be ventilated to provide oxygen to the subject. In particular, an endotracheal cuff of the endotracheal device may be inflated and a ventilator may be coupled to a ventilation port to ventilate the subject through the tube. The endotracheal cuff inflated during ventilation allows the airway to be sealed to maintain the position of the endotracheal device within the endotracheal system of the subject.

At block 525, the bronchoscope is retracted through the tube of the endotracheal device. In some examples, the bronchoscope may be retracted from the endotracheal device entirely. In other examples, the bronchoscope may be partially retracted through the tube of the endotracheal device. Preferably, the bronchoscope may be retracted through the tube sufficiently far so as to allow sufficient suction to be applied through the tube to engage the target obstruction. Thus, for example, the bronchoscope may be retracted past a suction port of the endotracheal device.

For example, referring to FIG. 6C, an example schematic diagram of the use of the endotracheal device 100 in the endotracheal system 600 at block 525 of the method 500 is depicted. In particular, after advancing the endotracheal device 100, and in particular, the tube 104, until the end surface 110 is positioned against the target obstruction 608, then the bronchoscope 604 is retracted through the tube 104 of the endotracheal device 100. In particular, the bronchoscope 604 is illustrated as being partially retracted in FIG. 6C. Further, as the bronchoscope 604 is retracted through the endotracheal device 100, the endotracheal device 100 maintains its position within the endotracheal system 600. That is, the end surface 110 is maintained in its position against the target obstruction. In some examples, an endotracheal cuff, such as the endotracheal cuff 212 of the endotracheal device 200, may be inflated to assist with maintaining the endotracheal device 100 at its position within the endotracheal system 600.

Returning again to FIG. 5, at block 530, the endotracheal device may be connected to a suction device to apply suction to the tube to engage the target obstruction at the end surface of the tube. For example, a suction port of the endotracheal device may be made accessible, for example either by uncapping or otherwise uncovering a suction port cap, or by using a port selector to select the suction port. The suction port may then be connected to the suction device to allow the suction device to apply suction to the tube through the suction port and the adaptor of the endotracheal device.

At block 535, while maintaining suction on the tube, the endotracheal device is retracted from the endotracheal system of the subject. By maintaining suction on the tube, the target obstruction is maintained engaged with the end surface of the tube, and is thereby extracted or removed together with the endotracheal device.

For example, referring to FIG. 6D, an example schematic diagram of the use of the endotracheal device 100 in the endotracheal system 600 at block 535 of the method 500 is depicted. In particular, after connecting the suction device to the endotracheal device 100, the endotracheal device 100 is retracted from the endotracheal system 600 while maintaining suction. Since the suction applied to the tube 104 engages the target obstruction 608 with the end surface 110, as the endotracheal device 100 is retracted from the endotracheal system 600, the target obstruction 608 is also extracted or removed from the endotracheal system 600 with the endotracheal device 100.

Accordingly, as described herein, an endotracheal device may be employed to remove an obstruction from the endotracheal system of a subject. The endotracheal device allows for a bronchoscope to be inserted therein to quickly and efficiently locate the target obstruction in the endotracheal system, thereby reducing the amount of time taken to remove the target obstruction. Further, to facilitate the insertion, advancement and retraction of the tube while reducing damage or irritation to the endotracheal system of the subject, the tube includes an end surface with at least one rounded edge, thereby creating a smooth surface with no sharp edges or protrusions.

The scope of the claims should not be limited by the embodiments set forth in the above examples but should be given the broadest interpretation consistent with the description as a whole.

1. An endotracheal device comprising:
a tube having an end surface at a first end of the tube, the end surface having a rounded edge and configured to be aligned with a target obstruction;
an adaptor disposed at a second end of the tube, the adaptor having:
a bronchoscope connector comprising an aperture configured to admit a bronchoscope through the second end along a length of the tube to the first end of the tube to guide the tube to the target obstruction; and
a suction port configured to connect to a suction device to apply suction to the tube to engage the target obstruction at the end surface.

2. The endotracheal device of claim 1, wherein the bronchoscope connector is further configured to allow retraction of the bronchoscope through the tube.

3. The endotracheal device of claim 1, wherein the tube further includes armoring configured to maintain a passageway through the length of the tube.

4. The endotracheal device of claim 1, further comprising a suction port cap configured to cover the suction port.

5. The endotracheal device of claim 1, wherein the adaptor further comprises a ventilation port configured to connect to a ventilator to ventilate through the length of the tube.

6. The endotracheal device of claim 5, further comprising a ventilation port cap configured to cover the ventilation port.

7. The endotracheal device of claim 5, further comprising a port selector configured to selectively cover one of the suction port or the ventilation port and allow access to an other of the suction port or the ventilation port.

8. The endotracheal device of claim 5, further comprising an endotracheal cuff configured to be inflated during ventilation via the ventilation port.

9. A method comprising:
administering an endotracheal device having an end surface with a rounded edge at a first end into an endotracheal system of a subject;
advancing a bronchoscope through the endotracheal device to a target obstruction in the endotracheal system of the subject;
advancing the endotracheal device along the bronchoscope to align the end surface against the target obstruction;
retracting the bronchoscope;
applying suction to the endotracheal device to engage the target obstruction; and
retracting the endotracheal device to remove the target obstruction from the endotracheal system of the subject.

10. The method of claim 9, wherein the bronchoscope is retracted past a suction port of the endotracheal device.

11. The method of claim 9, further comprising maintaining the suction applied to the endotracheal device while retracting the endotracheal device.

12. The method of claim 9, further comprising ventilating the subject through the endotracheal device prior to aligning the end surface of the endotracheal device against the target obstruction.

13. The method of claim 12, further comprising inflating an endotracheal cuff to maintain a position of the endotracheal device in the endotracheal system of the subject prior to ventilating the subject.
